(19) United States
(12) Patent Application Publication
(10) Pub. No.: US 2009/0277061 A1
(43) Pub. Date: Nov. 12, 2009

(54) Wrap Around Self Laminating Wristband

(75) Inventors: Sanjay K. Jain, St. Louis, MO (US); Mark Greer, O'Fallon, MO (US)

Correspondence Address: THOMPSON COBURN LLP, ONE US BANK PLAZA, SUITE 3500, ST LOUIS, MO, 63101, US

(73) Assignee: LASER BAND, LLC, St. Louis, MO (US)

(21) Appl. No.: 12/115,945

(22) Filed: May 6, 2008

(51) Int. Cl. A61B 5/117; G06Q 90/00; G09F 3/10; B42D 15/00

(52) U.S. Cl. 40/633; 705/500; 40/299.01; 283/74

(57) ABSTRACT

A wrap around, self laminating wristband comprises a substantially transparent laminate strap with an imaging area for receiving printed information and one or more layers of adhesive adjacent the imaging area so that as the strap is wrapped around itself to overlie the imaging area it laminates it. Alternate embodiments provide for a snap closure to secure the wristband instead of adhesive, forming the imaging area with a coating of a thermally sensitive or active print material, adding one or more labels for common use with the wristband, forming the wristband in a page of multiple wristbands or with labels or separately, forming the wristband as part of a printer processible business form, and providing the wristband forms either with or without labels in a continuous fan fold or roll format.

### BACKGROUND AND SUMMARY OF THE INVENTION

Self laminating wristbands have become widely available for medical applications and have achieved great commercial success principally through the inventive efforts of the assignee of the present invention. Examples of the several different embodiments of the self laminating wristbands that have received patents and which are experiencing great commercial success may be found in one or more of the following US patents, the disclosures of which are incorporated herein by reference: U.S. Pat. Nos. 5,933,993; 6,438,881; 7,017,293; and 7,222,448.

These self laminating wristbands are formed in part with a clamshell of laminating layers which are hinged together so that after separation of the wristband from the carrier the two clamshell halves are folded over to laminate an imaging portion. In almost all of these embodiments, the clamshells are hinged along their long dimension although at least one embodiment depicts the clamshell hinged at their ends. With this arrangement, the imaging area which is adhered to one of the clamshell halves is positioned to be readily laminated by merely folding over the two halves along the hinge joining them. This provides a quick and easy method for “assembling” or laminating the wristband after its separation from the carrier, and then applying it to the patient's wrist. Many millions of these design wristbands, in a number of different formats and arrangements, have been sold by the assignee, and customers/users have used them as they have found them to be ideally suited to their many applications.

In its continuing efforts to improve and develop alternate design wristbands, the inventor herein has developed for the assignee a new inventive design wrap around self laminating wristband which is elegantly simple, perhaps even simpler than the previous clamshell design, and which is arguably also faster to separate from the carrier and apply to the patient's wrist. In essence, this new invention comprises a slightly longer single width, substantially transparent strap having an imaging end for carrying the printed image and an opposite or “window” end for overlying the imaging end to laminate it. In other words, this design departs from the clamshell arrangement the assignee has commercialized to great success and instead provides a single strap which wraps around the patient's wrist with the opposite end providing a lay over see-through laminate or window area which is affixed in one of several alternative manners to attach the wristband to the wearer's wrist. While the entire strap may be provided as substantially transparent, as an alternative a decorative design may obscure a portion of the strap length to actually form a window through which the printed image may be viewed. In that embodiment, the window is preferably provided in a length appreciably longer than the imaging area to allow for adjustability in the amount of overlap, and thus wristband length, while ensuring that the imaging area is visible therethrough. While a “see-through” laminate is preferred, it is also possible that one or more cutouts could be provided to enhance the readability of a bar code, for example, or for any other purpose. Also, in some embodiments, the imaging area end may preferably be slightly smaller in width so that the strap can be wrapped around the wrist and the window end may be slightly misaligned or even be “loose” but yet completely cover and laminate the imaging area with its surface being entirely visible through the window. Yet another alternative is for the wristband to have a curved, contoured or curvilinear edge along at least a portion of at least one of its sides, maybe even to the extreme of an hourglass or “wasp-waisted” band formed along both edges of both sides, or to have contoured sides as exemplified by the disclosure found in co-pending application Ser. No. 11/553,873 filed Oct. 27, 2006, the disclosure of which is incorporated herein by reference

A number of alternate arrangements for the “wraparound” wristband of the present invention have been conceived of. For example, instead of providing a two ply form, a single ply laminate strap can be provided separately from a printed face stock label and the two assembled by adhering the label to the laminate strap before wrapping the strap around the patient's wrist. The label can be provided as self adhering and separate patches or stripes of adhesive be provided on the laminate strap, either to completely enclose the label as the wristband is secured or merely to attach the “wrapped” end to the opposite end so that while the label is captured between two outer layers of laminate it is not totally encapsulated by the overlying laminate strap. And, the adhesive may be applied on either side of the label so that the laminate strap may have its end either fixed or loose. For convenience, the word “laminate” or “laminating” is used herein to refer to an arrangement where two layers of laminate capture an imaging area therebetween, but not necessarily encapsulate it. In some instances the laminate layers may be adhered together to completely encapsulate the imaging area, as in most of the other self laminating wristband designs disclosed in the assignee's other patents. In other instances, however, the sides or the ends or even the sides and ends of the laminate overlay may not be affixed in place. For purposes of this disclosure, these arrangements shall also be considered as satisfying the “laminate” or “laminating” definition.

As this design wristband requires a “wrapping around” or overlap of one end over the other, the length of the wristband is preferably slightly longer than a corresponding “finished length” self laminating wristband of the clamshell design. This is not an issue should the wristband be intended for smaller wrists, such as for pediatric applications, and this design may be conveniently die cut into a standard 8½×11 inch sheet for laser printer processing. This design is also adaptable for adult sized wristbands for laser printing in a slightly longer sheet, such as legal size or 8½×14 inches. Nor is this an issue when the wristband business forms are provided in a continuous strip, either fan fold or roll, such as would be intended for thermal printing applications. As the most common format for wristbands is in combination with a plurality of labels, this design wristband is uniquely suited for thermal printers where the overall width of the form is limited by the throat size of the printer. Due to it being a single width, a wristband of the present invention would occupy the top half of a nominal two inch (2″) wide form and the bottom half would be occupied by a series of labels. Thus, a self laminating, adult sized, self laminating wristband along with a plurality of labels are conveniently provided in a continuous strip or roll format in two inch width suitable for processing by most thermal printers. As explained above, this design is versatile and suited to many formats.

The “stock” from which this design wristband would preferably be provided in would be the common two ply stock comprised of a face or imaging ply adhered with a pattern adhesive to a backing or laminate ply. The wristband would then be die cut into both of the face ply and the laminate ply: the imaging area die cut into the face ply and the strap die cut into the laminate ply. Depending on the pattern of the adhesive, for example should there be no adhesive joining the length of the strap and window to the face ply, the die cut could be an “interrupted” die cut with for example nicks in the die which would leave behind “ties” of both or at least one ply to hold at least the laminate strap in place before desired removal and use of the wristband. Then, the user would tear the strap loose along the interrupted die cut after printing. A “surround” of adhesive would preferably be applied around the image area, and would be exposed after separation of the wristband from the carrier so that it would adhere to the portion of the window end overlying the imaging area to “laminate” it by substantially encapsulating it in this embodiment.

As an alternate in the embodiments disclosed herein, instead of providing a label or face stock ply, the imaging area may be formed for a thermal printer version by applying an opaque white base coat followed by a thermal active coating and for increased durability a protective top coating if desired to a single laminate ply, either in the desired area or across the entire ply as determined by cost, raw stock, forming machine capabilities, etc. The coating material provided may be any chosen from a group of suitable materials chosen as would be known by those of ordinary skill in the art. Examples are Environmental Inks & Coatings EH012552 Direct Thermal coating, EC007094 Opaque white base coat and Organic Dyestuffs Corporation ORCO Black ODB-2 Leuco-dye thermal coat. This “stock” material could be used to form single wristband sheetlets, multiple wristband sheets, “combo” forms with labels in sheet format, and thermal printer formats comprising continuous strips in fan fold or roll format as well as any other format as exemplified in the various other patent filings owned by the assignee.

Yet another alternate embodiment incorporates mechanical fasteners to close the wristband about the wearer's wrist. One such example is a snap closure which would be located at each side of the “wrapped end” and two series of holes located along the strap edges inboard of the imaging area, one of which each snap closure would fasten to and join the strap ends. The length of the finished wristband would thus be variable depending on which pair of holes the snap closures were fastened to. In this embodiment, the laminate would overlie the imaging area but need not be sealed on any edge. As an alternative to including the mechanical fastener in the wristband, they may also be provided separately with the wristband having pre-punched holes or other areas of weakness to accommodate the assembly of the mechanical fastener after the wristband may be processed and printed.

While the principal advantages and features of the present invention have been briefly explained above, a fuller understanding may be gained by referring to the drawings and description of the preferred embodiments which follow.

### BRIEF DESCRIPTION OF THE DRAWINGS

FIG. 1 is a plan view of the wraparound wristband formed in a two ply carrier;

FIG. 2 is a bottom view of the wraparound wristband separated from the carrier and having a decorative imprint defining a window at the opposite end;

FIG. 3 is a plan view of a wraparound wristband with labels arranged in a strip orientation;

FIG. 4 is a perspective view of a roll of the wraparound wristband and label combo form depicted in FIG. 3;

FIG. 5 is a plan view of a wraparound wristband with labels arranged in a page format;

FIG. 6 is a plan view of a wraparound wristband in a carrier with a pair of snap closures and associated series of holes for attaching the wristband;

FIG. 7 is a plan view of the wraparound wristband with snap closures and a matrix of labels arranged in a page format;

FIG. 8 is a plan view of a plurality of wraparound wristbands with snap closures formed in a page format;

FIG. 9 is a plan view of a separated wraparound wristband having a pair of adhesive strips straddling an imaging area formed by a coating process;

FIG. 10 is a plan view of a plurality of wraparound wristbands as shown in FIG. 9 arranged in a page format; and

FIG. 11 is a plan view of a separated wraparound wristband having a single adhesive stripe at its end.

### DETAILED DESCRIPTION OF THE PREFERRED EMBODIMENTS

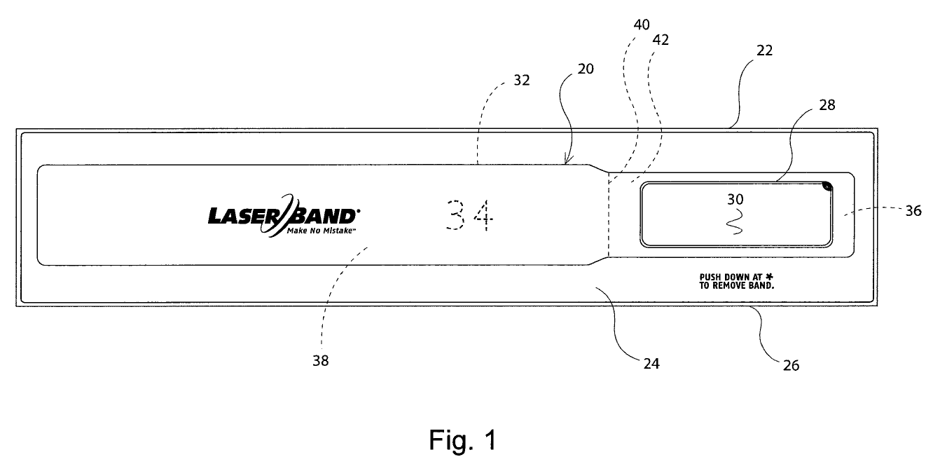

A first embodiment of the wraparound self laminating wristband 20 of the present invention is depicted in FIG. 1 and includes a two ply carrier 22 comprised of a top face ply 24 and a bottom laminate ply 26. A first die cut 28 in the face ply 24 forms an imaging area 30 and a second die cut 32 in the bottom laminate ply 26 forms a wristband strap 34. Die cut 32 may be an interrupted die cut, as depicted in other figures. The laminate wristband strap 34 is shown to preferably have a narrower end 36 whereat the imaging area 30 is adhered and a wider end 38. A dotted line 40 near the neck joining the narrower end 36 with the wider end 38 indicates the end of where a layer of adhesive 42 is applied to the narrower end 36 of wristband strap 34. A layer of release (not shown) surrounds the imaging area so that when the wristband 20 is separated from the carrier 22 it exposes the adhesive surrounding the imaging area 30. As there is preferably no release layer separating the laminate strap 32 from the imaging area 30, it remains adhered and separates with the laminate strap upon separation from the carrier 22 although it will separate with the laminate strap even if a release layer is applied if the directions for separating the wristband are followed. After separation by pushing down at the corner of the imaging area 30, the wristband 20 is applied to a wearer's wrist by wrapping the wider end 38 of laminate ply 34 about the wrist and pressing it over the top of the imaging area 30 to completely cover it and the surrounding layer of adhesive 42 adheres the two laminate layers together to laminate the imaging area 30. In this embodiment the wider end 36 helps allow for its inexact alignment over the imaging area 30 but which will still completely seal and encapsulate it. Any excess length may be trimmed off for comfort and to help guard against any patient discomfort from a floppy loose end.

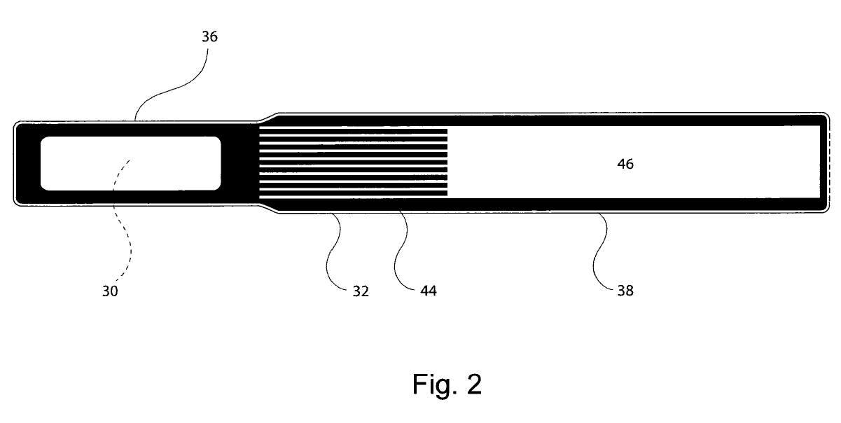

As shown in FIG. 2, the laminate strap 32 may be imprinted with a decorative pattern 44 to define a window 46 through which the imaging area 30 may be viewed. By making the window 46 substantially longer than the imaging area 30, the wristband may be sized to fit different wrists and yet ensure that a portion of the window 46 overlies the imaging area 30 to allow for its viewing. This is an alternative to providing the strap 32 as completely transparent.

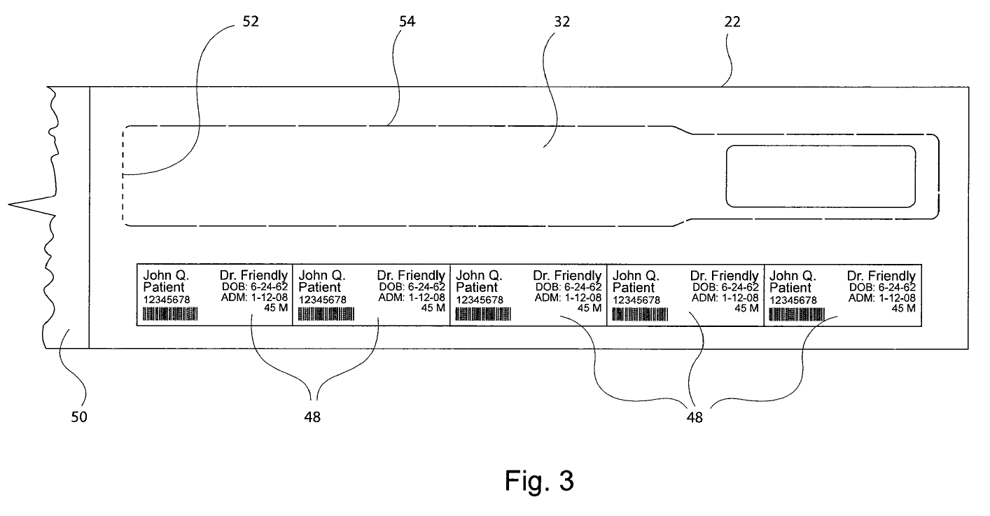

The wristband 20 may be provided in a continuous construction, with a series of labels 48 in a continuous strip 50 as depicted in FIG. 3. Also as shown in FIG. 3 is a line of perforation 52 at an end of the wristband laminate strap 32 which is of slightly different construction than the interrupted die cut line 54 also shown therein. The perforation line 52 provides slightly greater resistance to separation of the strap 32 from the carrier 22 than the interrupted die cut 54 and thus provides greater assurance that the strap 32 will not unintentionally separate during processing through a printer and other handling prior to use. With this increased strength, the perforation essentially forms a hinge. All of these variations are considered to be lines of weakness or die cuts, as those terms are used herein. Furthermore, the die cut to be used is to some extent a matter of design choice and depends on field testing in various applications with various materials, and the invention is not intended to be limited to the choice of die cut, etc. unless expressly so stated.

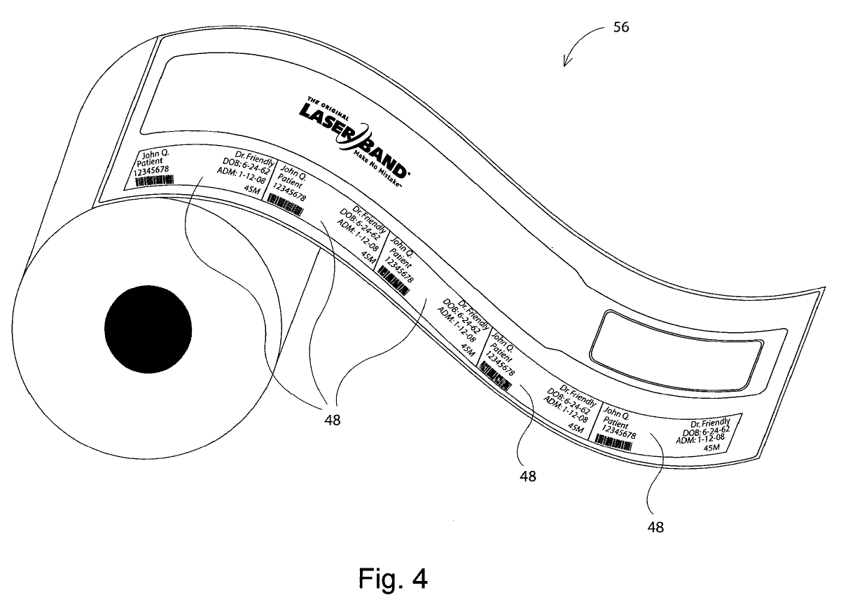

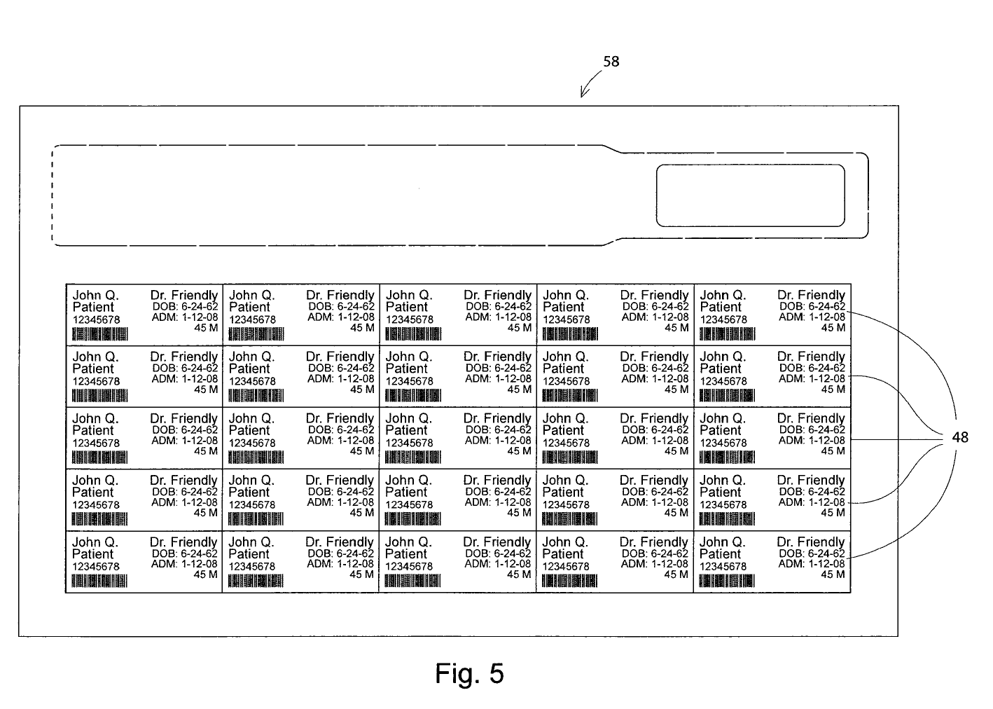

The continuous construction depicted in FIG. 3 may be provided in a fan fold configuration or in a roll format 56 as depicted in FIG. 4. Yet another format for use is as a page 58 as depicted in FIG. 5. In the formats depicted in FIG. 3-5, the web construction underlying the self adhering labels 48 may preferably be different, with a different backing sheet or web (not shown) having a release layer applied to it to expose the layer of adhesive applied to the labels 48 as they are removed from the carrier 22, as known in the art and as shown in various other patents owned by the assignee including those noted above.

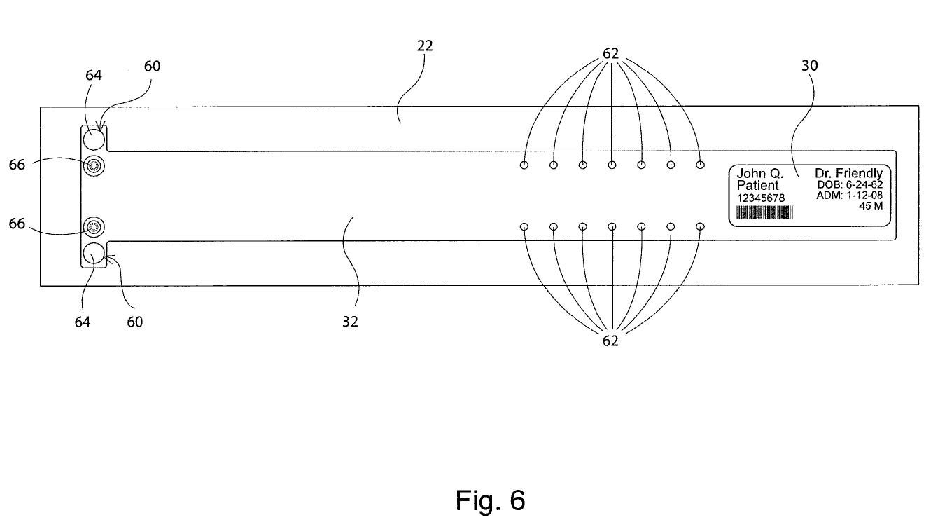

Instead of a layer of adhesive, one or more mechanical fasteners such as snap closures 60 each having an associated series of holes 62 may be used to attach the wristband about a wearer's wrist. Each snap closure may preferably include a male member 64 which inserts first through one of its associated holes 62 and then into a female member 66 to secure the wristband strap. When so fastened, one end of the laminate strap 32 overlies the imaging area 30 which may be a label separately supplied or provided as part of the wristband 20 formed as part of or on a carrier 22. In this embodiment the laminate strap 32 overlies the imaging area but preferably no adhesive is used so that the edges are not sealed, but nevertheless are laminated as that term is used herein. Should the wristband 20 shown in FIG. 6 be provided without a carrier 22, the strap 32 is preferably constructed of a somewhat heavier or thicker laminate and the imaging area 30 may preferably be a self adhering label.

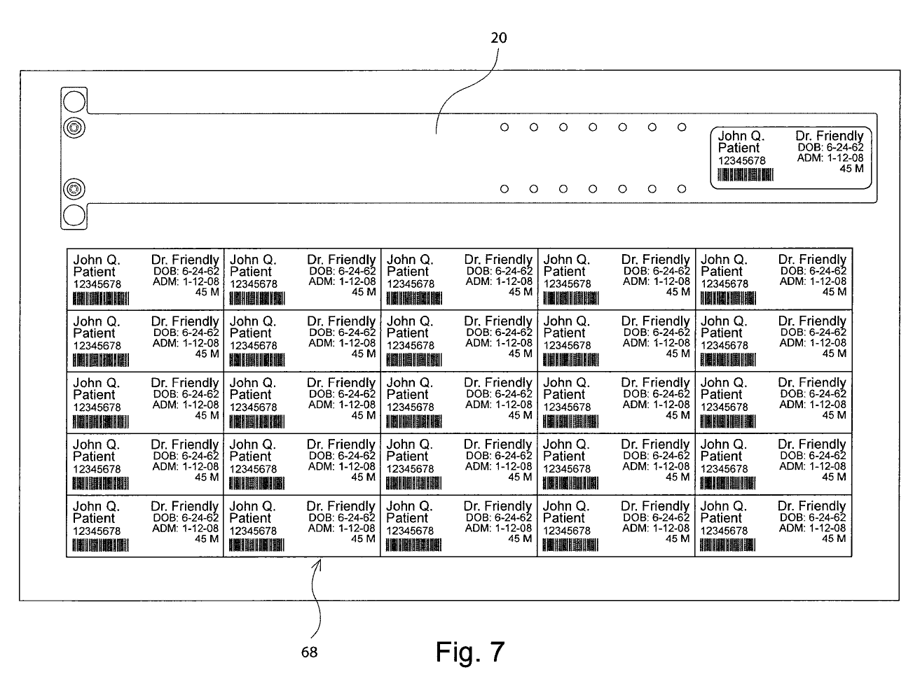

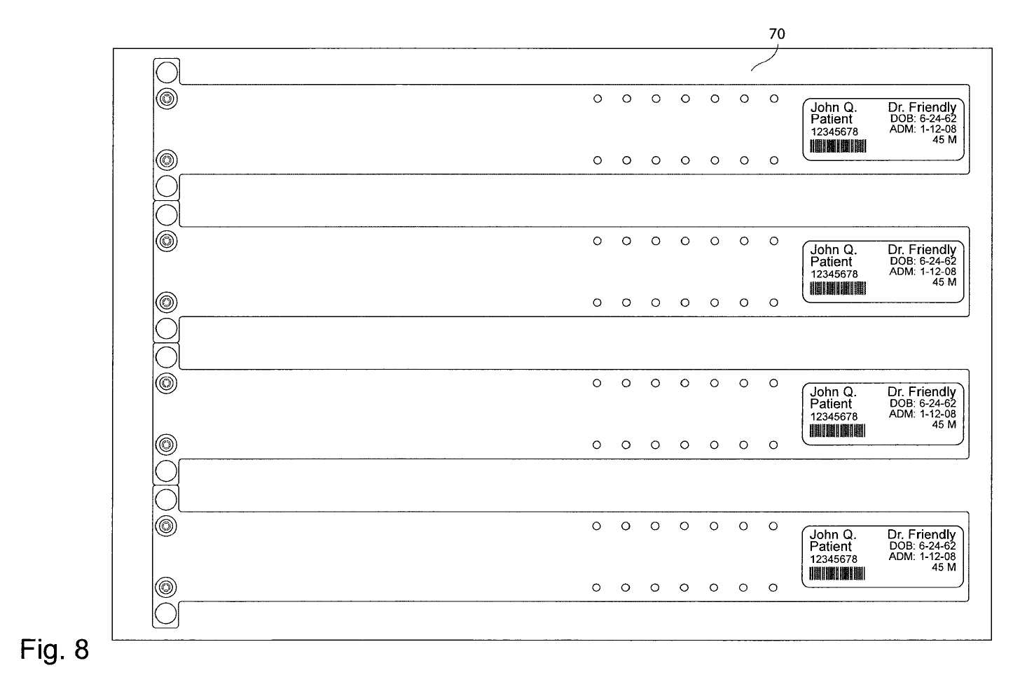

The wristband 20 as depicted in FIG. 6 may be combined and provided with a matrix of labels 68 in a page format as shown in FIG. 7, or provided as a group of wristbands only in a page 70 as shown in FIG. 8.

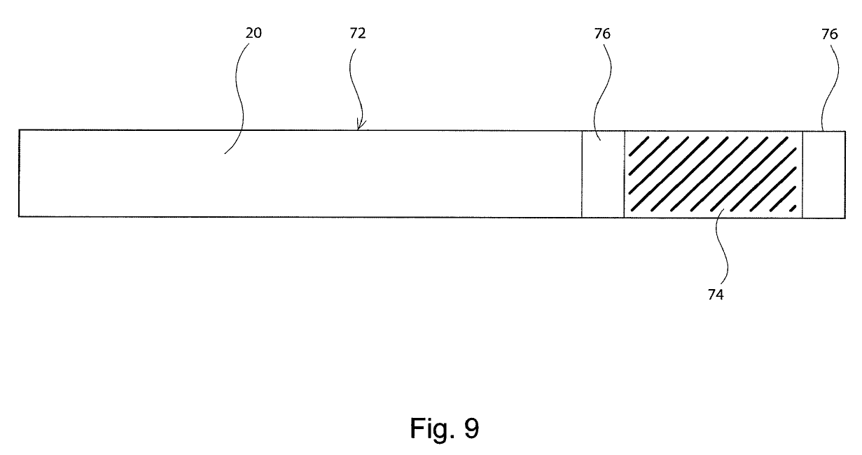

Yet another embodiment is depicted in FIG. 9 and includes a single laminate strap 72 with an imaging area 74 surrounded by a pair of adhesive patches or stripes 76. As depicted in FIG. 9, the wristband is essentially a strap and is formed by being die cut into a carrier, similar to that shown in FIG. 8 and described below. The strap may be thinner or thicker, as desired to suit the particular parameters of its intended use. Imaging area 74 may be a separately supplied and printed label, or may be a coating of a thermally receptive print material as described in greater detail above. To secure the wristband 20 of FIG. 9, the opposite end is wrapped around to overlie the two stripes of adhesive 76 and included imaging area 74 and pressed together. When properly adhered, the two ends of the imaging area 74 are sealed but not the sides, but nevertheless are laminated as that term is used herein.

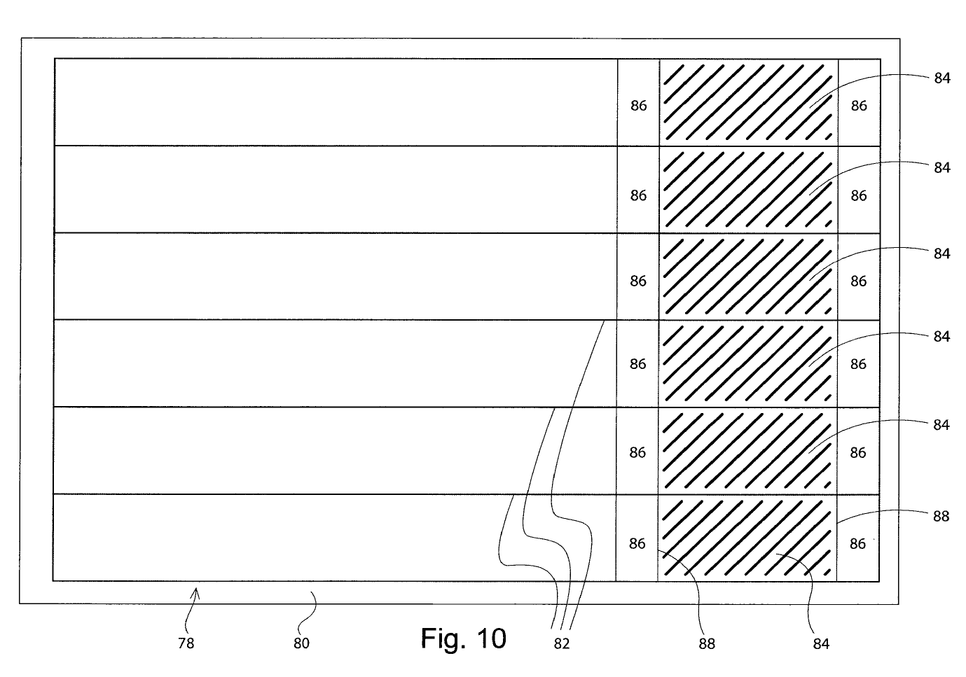

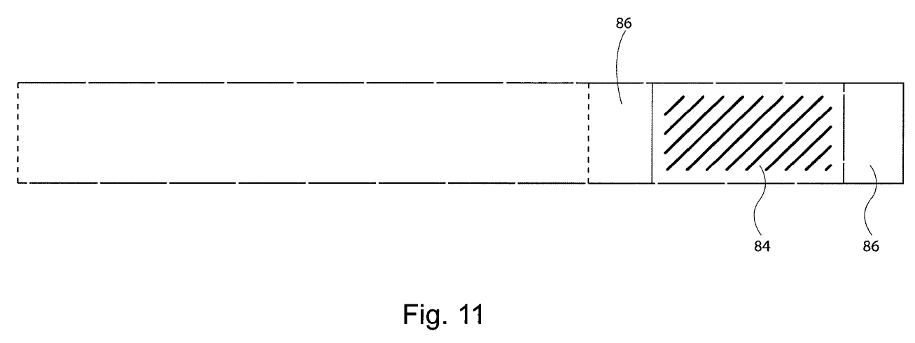

FIG. 10 depicts several variations for the wristband shown and described for FIG. 9. A plurality of wristbands 20 may comprise a page 78, either with or without a carrier 80. Adjacent wristbands 20 may be preferably separated by a line of weakness 82 so that they may be readily separated and individually used. An imaging area 84 is separately formed on each wristband 20 and is depicted as having a stripe of adhesive 86 on either side thereof, although alternatively a single stripe of adhesive may be provided on either side of the imaging area for each wristband as depicted in FIG. 11. The imaging area 84 may be formed from a coating of thermally receptive material and applied in a continuous fashion to the page of laminate prior to the die cuts 82 being made. Also, imaging areas 84 may be formed from a face stock ply adhered to the laminate ply comprising page ply 78. Further, the face stock ply used to form the imaging areas 84 may be readily extended to cover the adhesive stripes and die cuts 88 made to form removable tabs for exposing the adhesive stripes upon application to a wearer's wrist.

Various materials may be used as envisioned by the inventor. For example, polyethylene or Polyester or polypropylene may preferably be used for lighter and thinner laminate straps preferably having a thickness of about 1.5 to about 4 mil. For heavier and thicker laminates straps, Vinyl, polyethylene or polyurethane may preferably be used and preferably have a thickness of from about 4 to 12 mil. The face stock may preferably be standard #20 bond, or any other suitable paper stock as known in the art. Adhesives are also well known and their selection would be a matter of design choice depending on whether the wristband is intended to be processed through a laser or thermal printer. The imaging area may be formed by preferably applying a coating of EIC EH012552 or ORCO Black ODB-2 as a thermally receptive print material, preferably to a thickness of between about 0.1 to about 0.5 mil. These materials are all well known to those in the art and other suitable materials may be substituted therefor depending on the cost, availability, application and other design parameters.

While the invention has been disclosed in an illustrative manner through the drawing figures and description of the preferred embodiments, it would be apparent that other alternatives are included within the scope of the invention which should be limited solely by the scope of the appended claims and their equivalents. For example, other mechanical fasteners than snap closures may be used to fasten the wristband about the wearer's wrist. The imaging area may be formed as part of an overlying face stock ply or web, applied as a self adhering label after being printed with the desired information, applied as a thermally receptive print coating and then processed through a thermal printer, or otherwise. The laminate strap may have a substantially constant width along substantially the entirety of its length, have a narrower end at the imaging area, or have other shapes depending on the particular application. The laminate strap may be sized to overlap and be secured at intermediate locations allowing for it to be used on people having differently sized wrists. Thus, the laminate strap may have a variable length to suit the particular application. Adhesive stripes or patches may be applied in different locations, so long as they may be used to secure the wristband about the wearer's wrist. Although mention is made throughout this disclosure about a wearer's wrist, it should be understood that any wearer's appendage would be amenable for application of the present invention, and the term “wristband” has been used merely for convenience. Wristbands may be formed individually, in multiple numbers as part of a page of wristbands, combined with labels into “combo” forms, be formed in a reduced page size commonly referred to as a Sheetlet, or otherwise. Still other variations may be readily arrived at by those of skill in the art given the present teaching and these variations are intended to be included within the scope of this invention.

What is claimed is:

1. A printer processible business form comprising a self laminating wristband, said wristband comprising a laminate strap defined by a die cut in a laminate ply of said business form, and an imaging surface defined by a die cut in a face ply of said business form and adhered to an end of the laminate strap, a layer of adhesive applied to said laminate strap for securing the strap, and said laminate strap having a window near an end opposite said face ply end so that said wristband may be separated from said business form along said die cuts and wrapped around a wearer's wrist to adhere to said face ply end with the window overlying and laminating the imaging surface.

2. The business form of claim 1 wherein said laminate strap has contoured edges along at least a portion of its length.

3. The business form of claim 1 wherein said window has a length substantially longer than a length of the imaging surface to thereby allow the opposite end of the strap to wrap around the wearer's wrist to different lengths and yet still position the window substantially over the imaging surface.

4. The business form of claim 3 wherein said die cut defining the laminate strap comprises an interrupted die cut.

5. The business form of claim 4 wherein said strap has a portion thereof surrounding the imaging portion after its separation from the business form, said layer of adhesive being applied thereto for adhering the opposite end of said strap to thereby laminate the imaging portion and secure the wristband to the wearer's wrist.

6. The business form of claim 5 wherein the portion of said strap underlying the imaging portion has a width smaller than the portion of the strap containing the window.

7. The business form of claim 1 further comprising a plurality of labels, said wristband being formed in a first portion of said business form and the plurality of labels being formed in a second portion of said business form.

8. The business form of claim 7 wherein said first portion is atop the second portion, and a plurality of said business forms are arranged in a continuous strip.

9. The business form of claim 1 wherein said laminate strap has a curvilinear edge along at least one of its sides.

10. A printer processible business form comprising a wrap around, self laminating wristband, said wristband comprising a single width laminate strap having an integrally formed window at one end, an imaging portion at the opposite end, and an adhesive applied to said strap so that wrapping said window end around a wearer's wrist to overlie the imaging portion and adhere it thereto thereby laminates the imaging portion.

11. The business form of claim 10 wherein said window has a length substantially longer than the length of the imaging portion to thereby allow a differing length of the window end to overlap the imaging end and yet still align with the imaging portion as it is adhered in place.

12. The business form of claim 11 wherein the adhesive comprises a layer of adhesive applied to the strap surrounding the imaging area to thereby adhere the window end to the imaging end as the two ends are brought together about the wearer's wrist.

13. The business form of claim 12 wherein the strap is formed in a single ply of material.

14. The business form of claim 13 wherein the imaging portion comprises a coating of material applied to the single ply.

15. The business form of claim 13 wherein the imaging portion comprises a die cut portion of a second ply.

16. The business form of claim 10 further comprising a label portion die cut into said business form, said label portion forming at least one label.

17. The business form of claim 16 wherein the wristband is formed in a first portion thereof and the label portion is formed in a second portion thereof, and further comprising a plurality of said business forms arranged in a continuous strip.

18. The business form of claim 10 wherein said window comprises a clear laminate portion of said laminate strap.

19. A printer processible business form comprising a wrap around, self laminating wristband, said self laminating wristband comprising a single width laminate strap having an image receiving end and an opposing window end, and said strap being separable from said business form and further comprising a layer of adhesive applied to the image receiving end so that upon separation of said wristband from said business form and wrapping of the window end over the top of the image receiving end, the layer of adhesive adheres the two ends together to laminate an image applied to the image receiving end and secure the wristband to a wearer's wrist.

20. The business form of claim 19 wherein the image receiving end has an image receiving portion and the window end comprises a transparent strap portion, and wherein said transparent strap portion has a length appreciably longer than the image receiving portion so that the window end may be wrapped around the image receiving end to different positions and yet still align the transparent strap portion with the image receiving portion.

21. The business form of claim 20 wherein said layer of adhesive substantially completely surrounds the image receiving portion so that as the window end is adhered it laminates the image receiving portion by substantially completely encapsulating it.

22. The business form of claim 21 wherein the image receiving portion comprises a second layer of material adhered to said strap.

23. The business form of claim 22 wherein said image receiving end has a smaller width than the window end so that said window end, if properly aligned as the two ends are brought together to be adhered, completely covers the image receiving portion.

24. The business form of claim 19 further comprising a plurality of self adhering labels.

25. The business form of claim 23 further comprising a plurality of said forms joined in a continuous strip.

26. The business form of claim 24 further comprising a plurality of said forms joined in a continuous strip.

27. The business form of claim 19 wherein said wristband is formed by at least one die cut in said business form.

28. The business form of claim 27 wherein said die cut is an interrupted die cut.

29. A printer processible business form comprising a wrap around, self laminating wristband, said self laminating wristband comprising a single width laminate strap having an image receiving end and a wrapping end, and said strap being separable from said business form and further comprising a layer of adhesive applied to the image receiving end so that upon separation of said wristband from said business form and wrapping of the wrapping end over the top of the image receiving end, the layer of adhesive adheres the two ends together to laminate any image on the image receiving end and secure the wristband to a wearer's wrist.

30. The business form of claim 29 wherein said strap is substantially transparent at least at the wrapping end.

31. The business form of claim 30 wherein said strap is substantially transparent along substantially its entire length.

32. A substantially transparent laminate wristband strap, said strap having adhesive applied to at least one end thereof and being sufficiently long to wrap around a wearer's wrist and extend past the opposite end to affix the wristband on a wearer's wrist and overlie any imprinted data appearing on said opposite end.

33. The wristband strap of claim 32 further comprising a layer of imaging material applied to an end of said strap, said imaging material being adapted to receive and retain a printed image, and wherein said strap is substantially transparent so as to allow for viewing of the imprinted data lying underneath its opposite end.

34. The wristband strap of claim 33 wherein said adhesive comprises a patch of adhesive applied on at least one side of said imaging layer.

35. The wristband strap of claim 34 wherein said adhesive comprises a patch of adhesive applied to opposite sides of said imaging layer.

36. The wristband strap of claim 35 wherein said adhesive patches are substantially adjacent the imaging layer.

37. The wristband strap of claim 33 wherein said layer of imaging material comprises a label adhered to said strap.

38. The wristband strap of claim 33 wherein said layer of imaging material comprises a coating of thermally sensitive print material.

39. The wristband strap of claim 38 wherein said layer of imaging material is applied by spraying it onto the strap.

40. The wristband strap of claim 32 wherein a plurality of said straps are formed in a page with a line of weakness separating adjacent straps for easy separation therefrom and wherein at least some of said straps are substantially transparent so as to allow for viewing of the imprinted data when lying underneath its opposite end.

41. The wristband strap of claim 32 further comprising a self adhering label imprinted with information, said label being adhered to the strap opposite end, and wherein said strap is substantially transparent so as to allow for viewing of the imprinted data lying underneath its opposite end.

42. The wristband strap of claim 32 wherein said strap has a laminate layer and a face stock layer, said face stock layer being adapted to receive and retain printed information and positioned near the opposite end of said strap.

43. The wristband strap of claim 42 further comprising a carrier, said carrier having a face stock web and a laminate web, said face stock layer being defined by a die cut in said face stock web and said laminate layer being defined by a die cut in said laminate web, and wherein said face stock layer is adhered to said laminate layer.

44. A laminate wristband strap, said strap being adapted to receive a mechanical fastener attached near at least one end thereof and being sufficiently long to wrap around a wearer's wrist and extend past an opposite end to affix the wristband on a wearer's wrist and overlie any imprinted data appearing on said opposite end.

45. The wristband strap of claim 44 wherein said mechanical fastener comprises at least one snap closure affixed at an end of said strap and said strap is substantially transparent.

46. The wristband strap of claim 45 wherein said mechanical fastener further comprises a series of holes along said strap, said snap closure being adapted to affix to at least one of said holes to thereby affix the wristband about the wearer's wrist.

47. The wristband of claim 46 wherein said mechanical fastener further comprises a pair of snap closures adapted to be affixed at opposite sides of said strap, and a series of holes on opposite sides of said strap, each of said snap closures being aligned with one of said series of holes as the strap is wrapped around the wearer's wrist so that each of said snap closures may be affixed to one of the holes in its respective series of holes to thereby affix both sides of said strap in position.

48. The wristband of claim 47 further comprising a label affixed to the opposite end, said label being capable of receiving and retaining a printed image.

49. The wristband of claim 48 wherein said series of holes are inboard from the label so that affixing the snap closures to their respective hole affixes the strap over the label to thereby laminate it.

50. A business form comprising a substantially transparent laminate wristband strap, said strap having a mechanical fastener attached to at least one end thereof and being sufficiently long to wrap around a wearer's wrist and extend past an opposite end to affix the wristband on the wearer's wrist and overlie any imprinted data appearing on said opposite end, and a plurality of labels.

51. A business form comprising a page having a plurality of substantially transparent laminate wristband straps, at least some of said straps having a mechanical fastener attached to at least one end thereof and being sufficiently long to wrap around a wearer's wrist and extend past an opposite end to affix the wristband on a wearer's wrist and overlie any imprinted data appearing on said opposite end.

52. A substantially transparent laminate wristband strap, said strap having substantially the same width along its length, at least one stripe of adhesive applied near one end thereof for adhering that end to its opposite end to attach the wristband about a wearer's wrist, and a printer processible label applied to the adhesive end and located so that as said strap is wrapped around the wearer's wrist and adhered, a portion of said strap overlies said label.

53. The wristband of claim 52 wherein said at least one stripe of adhesive is applied the end of the strap.

54. The wristband of claim 52 wherein said at least one stripe of adhesive is applied inboard of the label.

55. The wristband of claim 52 wherein two stripes of adhesive are applied, with one of said stripes being located on each side of the label.
